(19) United States
(12) Patent Application Publication
(10) Pub. No.: US 2008/0279853 A1
(43) Pub. Date: Nov. 13, 2008

(54) TREATMENT OF CANCER

(75) Inventors: Linda C. Burkly, West Newton, MA (US); Jennifer Michaelson, Brighton, MA (US)

Correspondence Address: BIOGEN IDEC / FINNEGAN HENDERSON, LLP, 901 NEW YORK AVENUE, NW, WASHINGTON, DC, 20001-4413, US

(73) Assignee: Biogen Idec MA Inc., Cambridge, CA (US)

(21) Appl. No.: 11/944,714

(22) Filed: Nov. 26, 2007

Related U.S. Application Data
(63) Continuation of application No. PCT/US2006/020262, filed on May 25, 2006.
(60) Provisional application No. 60/685,465, filed on May 27, 2005.

(51) Int. Cl. A61K 39/395; A61P 35/00

(52) U.S. Cl. 424/135.1; 424/174.1; 424/133.1

(57) ABSTRACT

Methods of treating cancer using an agent that blocks interaction between TWEAK and its receptor are described.

### CROSS-REFERENCE TO RELATED APPLICATIONS

This application, filed under 35 U.S.C. § 111, is a continuation claiming priority under 35 U.S.C. § 120 of International Application No. PCT/US2006/020262, filed on May 25, 2006, which claims priority to U.S. Application Ser. No. 60/685,465, filed on May 27, 2005. The contents of all the foregoing applications are hereby incorporated by reference.

### BACKGROUND OF THE INVENTION

Tweak (TNF-like weak inducer of apoptosis) is a member of the tumor-necrosis factor (TNF) superfamily of cytokines. Tweak induces diverse cellular responses in various systems. Tweak has been reported to promote apoptosis and/or necrosis of certain tumor cell lines, including HT29 colon adenocarcinoma cells (Chicheportiche et al., 1997, J Biol Chem. 272(51):32401-10; Nakayama et al., 2002, The Journal of Immunology, 168:734-743). Tweak has also been reported to stimulate angiogenesis when overexpressed in embryonic kidney cells (Ho et al., 2004, Cancer Res 64:8968-8972). Tweak has also been reported to play a substantial role in macrophage-mediated anti-tumor response in vivo and it has been reported that anti-Tweak antibodies promote the growth of Tweak receptor (TWEAK-R) (also known as Fn14)-expressing leukemia cells in vivo (Kaduka et al., 2005, Biochem Biophys Res Comm 331:384-390).

### SUMMARY OF THE INVENTION

Targeting of the Tweak pathway (e.g., with a Tweak pathway inhibitor such as an agent that blocks a Tweak-TweakR interaction, e.g., an anti-Tweak or anti-Fn14 blocking antibody) has been found to be effective in treatment of certain cancers (e.g., carcinomas, such as adenocarcinoma) in vivo. Accordingly, the invention features methods and compositions useful in the treatment of cancer.

In one aspect, the invention features a method for treating a cancer in a subject, such as a human. The method includes administering, to the subject, an agent that blocks a Tweak/Tweak-R interaction or activity. In one embodiment, the agent is an antibody, e.g., a Tweak or Fn14 antibody, or a soluble form of Tweak receptor, e.g., a soluble Fn14.

In one embodiment, the cancer is a solid cancer, e.g., a carcinoma, e.g., an adenocarcinoma. The cancer, carcinoma or adenocarcinoma can be selected from pancreatic, breast, lung, prostate, colon, colorectal, skin, ovarian, cervical, or renal cancer, carcinoma or adenocarcinoma. In some embodiments, the cancer, carcinoma or adenocarcinoma is metastatic, advanced and/or late-stage (e.g., stage III or later). In other embodiments, the cancer is stage II or earlier. The cancer, carcinoma or adenocarcinoma may be Fn14-positive or it may overexpress Fn14 relative to a non-cancer tissue.

In one embodiment, the agent is an antibody that is a full length IgG. In other embodiments, the agent is an antigen-binding fragment of a full length IgG, e.g., the agent is a single chain antibody, Fab fragment, F(ab′)2 fragment, Fd fragment, Fv fragment, or dAb fragment. In preferred embodiments, the antibody is a human, humanized or humaneered antibody or antigen-binding fragment thereof. In one embodiment, the antibody is a humanized P2D10 antibody.

In one embodiment, the agent is a soluble form of the Tweak receptor, e.g., a polypeptide at least 95% identical to amino acids 28-X1 of SEQ ID NO:2, where amino acid X1 is selected from the group of residues 68 to 80 of SEQ ID NO:2. In some cases, the soluble form of the receptor is fused with a heterologous polypeptide, e.g., an antibody Fc region.

In one embodiment, the agent is administered in an amount sufficient to do one or more of the following: (a) reduce tumor growth rate, (b) inhibit tumor metastasis, (c) reduce tumor size, (d) increase the subject's body weight or inhibit body weight loss, (e) reduce tumor survival, (f) reduce tumor invasiveness, e.g., reduce invasiveness into local organs, (g) increase sensitivity to a chemotherapeutic agent, (h) reduce dose of a chemotherapeutic agent being co-administered, (i) inhibit angiogenesis of the tumor, (j) reduce one or more clinical symptoms in the subject (e.g., reduce pain or nausea), (k) increase survival of the subject, e.g., increase the time from diagnosis to death, (l) reduce progression of the cancer, e.g., reduce time of progression from stage I to stage II, from stage II to stage III, and so on. Such increases or reductions can be, e.g., increases or reductions of at least 10%, 20%, 30%, 40%, 50%, 60% or more compared to a reference value.

In one embodiment, the subject has an adenocarcinoma, e.g., a pancreatic adenocarcinoma.

In one embodiment, the agent is administered in combination with another therapy for the cancer, e.g., a chemotherapy or radiotherapy. The chemotherapy can be, e.g., 5FU and/or gemcitabine. In one embodiment, the other therapy is another biologic therapy, e.g., an anti-VEGF antibody (e.g., bevacizumab).

In one embodiment, the agent is administered at a dose between 0.1-100 mg/kg, between 0.1-10 mg/kg, between 1 mg/kg-100 mg/kg, between 0.5-20 mg/kg, or between 1-10 mg/kg. In the most typical embodiment, the dose is administered more than once, e.g., at periodic intervals over a period of time (a course of treatment). For example, the dose may be administered every 2 months, every 6 weeks, monthly, biweekly, weekly, or daily, as appropriate, over a period of time to encompass at least 2 doses, 3 doses, 5 doses, 10 doses, or more.

In one embodiment, the invention features an agent that blocks a Tweak/Tweak-R interaction or activity, wherein the agent is an antibody or a soluble form of Tweak receptor, for use in therapy, e.g., cancer therapy.

In some embodiments, the invention features use of an agent that blocks a Tweak/Tweak-R interaction or activity, wherein the agent is an antibody or a soluble form of Tweak receptor, for the preparation of a medicament for the treatment of cancer, e.g., pancreatic, breast, lung, prostate, colon, colorectal, skin, and renal cancer, in a subject.

The term “treating” refers to administering a therapy in an amount, manner, and/or mode effective to improve or prevent a condition, symptom, or parameter associated with a disorder or to prevent onset, progression, or exacerbation of the disorder (including secondary damage caused by the disorder), to either a statistically significant degree or to a degree detectable to one skilled in the art. Accordingly, treating can achieve therapeutic and/or prophylactic benefits. An effective amount, manner, or mode can vary depending on the subject and may be tailored to the subject.

Each of the limitations described herein can encompass various embodiments of the invention. It is, therefore anticipated that each of the limitations of the invention involving any one element or combinations of elements can be included in each aspect of the invention.

### BRIEF DESCRIPTION OF THE DRAWINGS

FIG. 1 is a bar graph showing results from an invasion assay of human pancreatic cell lines with and without added Tweak (Tk), and in the presence or absence of an anti-Tweak antibody (p5g9). HGF is a positive control.

FIG. 2 is a graph showing growth of human pancreatic tumor cell line BxPc-3 in a xenograft animal model, with and without anti-Tweak antibody (P2D10) treatment.

### DETAILED DESCRIPTION OF THE INVENTION

It has been found that agents that block the Tweak pathway, e.g., by blocking Tweak/Tweak-R interaction, can be used to treat a cancer, e.g., a carcinoma, e.g., an adenocarcinoma such as a pancreatic adenocarcinoma.

Exemplary information regarding the diagnosis and prognosis of pancreatic cancer is provided below. However, it is understood that methods of identifying and diagnosing other types of cancers, e.g., cancers described herein, are known to a practitioner of ordinary skill.

### Pancreatic Cancer

Early diagnosis of pancreatic cancer is difficult because of the retroperitoneal location of the pancreas and the fact that pancreatic cancer does not typically cause symptoms in its early stages. At the time that symptoms occur, the tumor may often already have metastasized. Symptoms include abdominal pain, nausea, loss of appetite, weight loss, digestive problems, and jaundice. The rarer endocrine pancreatic cancers may also cause restlessness, loss of energy, irritability, sweating, tremor, drowsiness and severe confusion.

Because the symptoms are general in nature, multiple diagnostic tools are frequently used to diagnose pancreatic cancer. These include ultrasound, computed tomography (CT) scan, MRI, barium meal ERCP (endoscopic retrograde cholangiopancreatography) and PTC (percutaneous transhepatic cholangiopancreatography) tests. Fine needle aspiration for tissue biopsy is also used in pancreatic cancer diagnosis. Biomarkers also have some use in diagnosis. For example, carbohydrate antigen 19-9 (CA 19-9) may be used to support a diagnosis of cancer, when high levels are measured, but it is not specific to pancreatic cancer. Carcinoembryonic antigen (CEA), serum amylase, alkaline phosphatase and bilirubin have been mentioned as being useful tests in the diagnosis of pancreatic cancer in select patients, but they are not universally specific or sensitive in the general population.

The staging of pancreatic cancer is based on the revised criteria of TNM staging by the American Joint Committee for Cancer (AJCC) (Table 1, Source: AJCC Cancer Staging manual, 5th ed., American Joint Committee on Cancer, Lippincott-Raven: Philadelphia; 1997:121-4). Tumors are assessed by the size of the primary tumor (T); the degree to which regional lymph nodes (N) are involved; and the absence or presence of metastases (M). A “stage” of I, II, III or IV is then assigned. Stage I cancers are small, localized and usually curable. Stage II and III cancers typically are locally advanced and/or have spread to local lymph nodes. Stage 1V cancers usually are metastatic and generally are considered inoperable.

TABLE 1
Pancreatic Cancer TNM Definitions and Stage Groupings

| T | Primary Tumor | |
|---|---|---|
| | TX | Primary tumor cannot be assessed |
| | T0 | No evidence of primary tumor |
| | Tis | In situ carcinoma |
| | T1 | Tumor limited to the pancreas, 2 cm or less in |
| | | greatest dimension |
| | T2 | Tumor limited to the pancreas, >2 cm in greatest |
| | | dimension |
| | T3 | Tumor extends directly into duodenum, bile duct, or |
| | | peripancreatic tissues |
| | T4 | Tumor extends directly into stomach, spleen, colon, |
| | | or adjacent large vessels |
| N | Regional Lymph Nodes | |
| | NX | Regional lymph nodes cannot be assessed |
| | N0 | No regional lymph nodemetastasis |
| | N1 | Regional lymph node metastasis (pNla—Metastasis |
| | | in a single regional lymph node; |
| | | pNIb—Metastasis in multiple regional lymph nodes) |
| M | Distant Metastasis | |
| | MX | Distant metastasis cannot be assessed |
| | M0 | No distant metastasis |
| | M1 | Distant metastasis |

| | Stage Grouping | | |
|---|---|---|---|
| Stage 0 | Tis | N0 | M0 |
| Stage I | T1 | N0 | M0 |
| | T2 | N0 | M0 |
| Stage II | T3 | N0 | M0 |
| Stage III | T1 | N1 | M0 |
| | T2 | N1 | M0 |
| | T3 | N1 | M0 |
| Stage IVA | T4 | Any N | M0 |
| Stage IMB | Any T | Any N | M1 |

More than 90% of pancreatic malignancies arise from ductal epithelium, even though less than 15% of the pancreas by mass is made up of ductal tissue. At least 90% of pancreatic cancers are adenocarcinomas, usually originating in the head of the gland. Other tumors arising from the pancreas include acinar cell carcinoma (about 5%), cystadenocarcinoma (mucinous), adenosquamous carcinoma, solid microglandular carcinoma, carcinoid, sarcoma, and malignant lymphoma. Cancers arising in the head of the pancreas must be distinguished from peripancreatic lesions arising from the distal common bile duck, the ampulla of Vater, or the duodenum. While ampullary cancer is the most resectable and associated with the most favorable prognosis, survival rates with all three are higher than with pancreatic cancer.

Advanced age and smoking are risk factors for pancreatic cancer. Exposure to certain petroleum, chemical and metal products may also correlate with increased risk. Excessive dietary fat and protein combined with low fiber intake may promote the disease.

Current treatment options for pancreatic cancer include surgery to remove the tumor mass, radiotherapy, and chemotherapy. Exemplary chemotherapeutic agents include fluorouracil (5-FU) and gemcitabine.

### Tweak/Tweak Receptor Blocking Agents

Tweak pathway inhibitors to treat pancreatic cancer include Tweak/Tweak-R blocking agents. The agent may be any type of compound (e.g., small organic or inorganic molecule, nucleic acid, protein, or peptide mimetic) that can be administered to a subject.

In one embodiment, the blocking agent is a biologic, e.g., a protein having a molecular weight of between 5-300 kDa. For example, a Tweak/Tweak-R blocking agent may inhibit binding of Tweak to a Tweak-R. A typical Tweak/Tweak-R blocking agent can bind to Tweak or a Tweak-R, e.g., Fn14. A Tweak/Tweak-R blocking agent that binds to Tweak or Tweak-R may block the binding site on Tweak or a Tweak receptor, alter the conformation of Tweak or a Tweak receptor, or otherwise decrease the affinity of Tweak for a Tweak-R or prevent the interaction between Tweak and a Tweak-R. A Tweak/Tweak-R blocking agent (e.g., an antibody) may bind to Tweak or to a Tweak-R with a Kd of less than 10⁻⁶, 10⁻⁷, 10⁻⁸, 10⁻⁹, or 10⁻¹⁰ M. In one embodiment, the blocking agent binds to Tweak with an affinity at least 5, 10, 20, 50, 100, 200, 500, or 1000-fold better than its affinity for TNF or another TNF superfamily member (other than Tweak). In one embodiment, the blocking agent binds to the Tweak-R with an affinity at least 5, 10, 20, 50, 100, 200, 500, or 1000-fold better than its affinity for the TNF receptor or a receptor for another TNF superfamily member. Exemplary Tweak/Tweak-R blocking agents include antibodies (e.g., monoclonal antibodies) that bind to Tweak or Tweak-R and soluble forms of the Tweak-R that compete with cell surface Tweak-R for binding to Tweak.

The sequence of an exemplary human Tweak is shown as SEQ ID NO:1:

```
    1 maarrsqrrr grrgepgtal lvplalglgl alaclgllla vvslgsrasl saqepaqeel
  61 vaeedqdpse lnpqteesqd papflnrlvr prrsapkgrk trarraiaah yevhprpgqd
121 gaqadggytt clrp
```

Also included are proteins that include an amino acid sequence at least 90, 92, 95, 97, 98, or 99% identical, or completely identical, to the mature processed region of SEQ ID NO:1 (e.g., an amino acid sequence at least 90, 92, 95, 97, 98, or 99% identical, or completely identical, to amino acids X1-249 of SEQ ID NO:1, where amino acid X1 is selected from the group of residues 75-115 of SEQ ID NO:1, e.g., X1 is residue Arg 93 of SEQ ID NO:1) and proteins encoded by a nucleic acid that hybridizes under high stringency conditions to the human nucleic acid sequence encoding SEQ ID NO:1. Preferably, a Tweak protein, in its processed mature form, is capable of providing at least one Tweak activity, e.g., ability to activate Fn14 and/or cell death in cortical neurons.

The sequence of an exemplary human Tweak-R is shown as SEQ ID NO:2:

```
    1 margslrrll rllvlglwla llrsvageqa pgtapcsrgs swsadldkcm dcascrarph
  61 sdfclgcaaa ppapfrllwp ilggalsltf vlgllsgflv wrrcrrrekf ttpieetgge
121 gcpavaliq
```

A soluble Tweak-R includes proteins that include an amino acid sequence at least 90, 92, 95, 97, 98, or 99% identical, or completely identical, to the extracellular domain of Fn14 (and Tweak-binding fragments thereof) and proteins encoded by nucleic acids that hybridize under high stringency conditions to human Tweak-R. Preferably, a Tweak-R protein useful in the methods described herein is a soluble Fn14 (lacking a transmembrane domain) that includes a region that binds to a Tweak protein, e.g., an amino acid sequence at least 90, 92, 95, 97, 98, or 99% identical, or completely identical, to amino acids 28-X1 of SEQ ID NO:2, where amino acid X1 is selected from the group of residues 68 to 80 of SEQ ID NO:2.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

As used herein, the term “hybridizes under high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. High stringency hybridization conditions include hybridization in 6×SSC at about 45° C., followed by one or more washes in 0.2×SSC, 0.1% SDS at 65° C., or substantially similar conditions.

Exemplary Tweak/Tweak-R blocking agents include antibodies that bind to Tweak or Tweak-R and soluble forms of the Tweak-R that compete with cell surface Tweak-R for binding to Tweak. An example of a soluble form of the Tweak-R is an Fc fusion protein that includes at least a portion of the extracellular domain of Tweak-R (e.g., a soluble Tweak-binding fragment of Tweak-R), referred to as Tweak-R-Fc. Other soluble forms of Tweak-R, e.g., forms that do not include an Fc domain, can also be used. Antibody blocking agents are further discussed below. Other types of blocking agents, e.g., small molecules, nucleic acid or nucleic acid-based aptamers, and peptides, can be isolated by screening, e.g., as described in Jhaveri et al. Nat. Biotechnol. 18:1293 and U.S. Pat. No. 5,223,409. Exemplary assays for determining if an agent binds to Tweak or Tweak-R and for determining if an agent modulates a Tweak/Tweak-R interaction are described, e.g., in U.S. 2004-0033225.

An exemplary soluble form of the Tweak-R protein includes a region of the Tweak-R protein that binds to Tweak, e.g., about amino acids 32-75, 31-75, 31-78, or 28-79 of GENBANK® NP_{—}057723.1. This region can be physically associated, e.g., fused to another amino acid sequence, e.g., an Fc domain, at its N- or C-terminus. The region from Tweak-R can be spaced by a linker from the heterologous amino acid sequence. U.S. Pat. No. 6,824,773 describes an exemplary Tweak-R fusion protein.

### Antibodies

Exemplary Tweak/Tweak-R blocking agents include antibodies that bind to Tweak and/or Tweak-R. In one embodiment, the antibody inhibits the interaction between Tweak and a Tweak receptor, e.g., by physically blocking the interaction, decreasing the affinity of Tweak and/or Tweak-R for its counterpart, disrupting or destabilizing Tweak/Tweak-R complexes, sequestering Tweak or a Tweak-R, or targeting Tweak or Tweak-R for degradation. In one embodiment, the antibody can bind to Tweak or Tweak-R at one or more amino acid residues that participate in the Tweak/Tweak-R binding interface. Such amino acid residues can be identified, e.g., by alanine scanning. In another embodiment, the antibody can bind to residues that do not participate in the Tweak/Tweak-R binding. For example, the antibody can alter a conformation of Tweak or Tweak-R and thereby reduce binding affinity, or the antibody may sterically hinder Tweak/Tweak-R binding. In one embodiment, the antibody can prevent activation of a Tweak/Tweak-R mediated event or activity (e.g., NF-κB activation).

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)2 fragments, Fd fragments, Fv fragments, and dAb fragments) as well as complete antibodies, e.g., intact and/or full length immunoglobulins of types IgA, IgG (e.g., IgG1, IgG2, IgG3, IgG4), IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A., et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two beta-sheets formed of about seven beta-strands, and a conserved disulphide bond (see, e.g., A. F. Williams and A. N. Barclay (1988) Ann. Rev Immunol. 6:381-405). An “immunoglobulin variable domain sequence” refers to an amino acid sequence that can form a structure sufficient to position CDR sequences in a conformation suitable for antigen binding. For example, the sequence may include all or part of the amino acid sequence of a naturally occurring variable domain. For example, the sequence may omit one, two or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with Tweak or a Tweak receptor.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2, and CH3. The light chain constant region typically includes a CL domain. The variable region of the heavy and light chains contain a binding domain that interacts with an antigen. The constant regions of the antibodies typically mediate the binding of the antibody to host tissues or factors, including various cells of the immune system (e.g., effector cells) and the first component (C1q) of the classical complement system.

One or more regions of an antibody can be human, effectively human, or humanized. For example, one or more of the variable regions can be human or effectively human. For example, one or more of the CDRs, e.g., HC CDR1, HC CDR2, HC CDR3, LC CDR1, LC CDR2, and LC CDR3, can be human. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical, or completely identical, to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762. In some cases, humanized immunoglobulins can include a non-human amino acid at one or more framework amino acid positions. Exemplary humanized antibodies that bind to TWEAK are described below as huP2D10-1 and huP2D10-2.

### Antibody Generation

Antibodies that bind to Tweak or a Tweak-R can be generated by a variety of means, including immunization, e.g., using an animal, or in vitro methods such as phage display. All or part of Tweak or a Tweak receptor can be used as an immunogen or as a target for selection. For example, Tweak or a fragment thereof, Tweak-R or a fragment thereof, can be used as an immunogen. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nat. Gen. 7:13-21; U.S. 2003-0070185; U.S. Pat. No. 5,789,650; and WO 96/34096.

Non-human antibodies to Tweak or a Tweak receptor can also be produced, e.g., in a rodent. The non-human antibody can be humanized, e.g., as described in EP 239 400; U.S. Pat. Nos. 6,602,503; 5,693,761; and 6,407,213, deimmunized, or otherwise modified to make it effectively human.

EP 239 400 (Winter et al.) describes altering antibodies by substitution (within a given variable region) of their complementarity determining regions (CDRs) for one species with those from another. Typically, CDRs of a non-human (e.g., murine) antibody are substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody. Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and 6,407,213; Tempest et al. (1991) Biotechnology 9:266-271.

Fully human monoclonal antibodies that bind to Tweak or a Tweak receptor can be produced, e.g., using in vitro-primed human splenocytes, as described by Boerner et al. (1991) J. Immunol. 147:86-95. They may be prepared by repertoire cloning as described by Persson et al. (1991) Proc. Nat. Acad. Sci. USA 88:2432-2436 or by Huang and Stollar (1991) J. Immunol. Methods 141:227-236; also U.S. Pat. No. 5,798,230. Large nonimmunized human phage display libraries may also be used to isolate high affinity antibodies that can be developed as human therapeutics using standard phage technology (see, e.g., Hoogenboom et al. (1998) Immunotechnology 4:1-20; Hoogenboom et al. (2000) Immunol. Today 2:371-8; and U.S. 2003-0232333).

### Antibody and Protein Production

Antibodies and other proteins described herein can be produced in prokaryotic and eukaryotic cells. In one embodiment, the antibodies (e.g., scFv's) are expressed in a yeast cell such as Pichia (see, e.g., Powers et al. (2001) J. Immunol. Methods 251:123-135), Hanseula, or Saccharomyces.

Antibodies, particularly full length antibodies, e.g., IgG's, can be produced in mammalian cells. Exemplary mammalian host cells for recombinant expression include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NSO myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). Exemplary selectable marker genes include the dihydrofolate reductase (DHFR) gene (for use in dhfr-host cells with methotrexate selection/amplification) and the neo gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies (and Fc fusions) may also include modifications, e.g., modifications that alter Fc function, e.g., to decrease or remove interaction with an Fc receptor or with C1q, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260. Other IgG constant regions can include like modifications.

For some proteins that include an Fc domain, the antibody/protein production system may be designed to synthesize antibodies or other proteins in which the Fc region is glycosylated. For example, the Fc domain of IgG molecules is glycosylated at asparagine 297 in the CH2 domain. The Fc domain can also include other eukaryotic post-translational modifications. In other cases, the protein is produced in a form that is not glycosylated.

Antibodies and other proteins can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the protein of interest, e.g., an antibody or Fc fusion protein. The protein can be purified from the milk, or for some applications, used directly.

Methods described in the context of antibodies can be adapted to other proteins, e.g., Fc fusions and soluble receptor fragments.

### Nucleic Acid Antagonists

In certain implementations, nucleic acid antagonists are used to decrease expression of an endogenous gene encoding Tweak or a Tweak-R, e.g., Fn14. In one embodiment, the nucleic acid antagonist is an siRNA that targets mRNA encoding Tweak or a Tweak-R. Other types of antagonistic nucleic acids can also be used, e.g., a dsRNA, a ribozyme, a triple-helix former, or an antisense nucleic acid.

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). See, e.g., Clemens et al. (2000) Proc. Natl. Acad. Sci. USA 97:6499-6503; Billy et al. (2001) Proc. Natl. Acad. Sci. USA 98:14428-14433; Elbashir et al. (2001) Nature 411:494-498; Yang et al. (2002) Proc. Natl. Acad. Sci. USA 99:9942-9947, U.S. 2003-0166282, 2003-0143204, 2004-0038278, and 2003-0224432.

Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA (e.g., an mRNA encoding Tweak or Tweak-R) can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid, e.g., the mRNA encoding Tweak or Tweak-R. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N4-(C1-C12) alkylaminocytosines and N4,N4-(C1-C12) dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N6-(C1-C12) alkylaminopurines and N6,N6-(C1-C12) dialkylaminopurines, including N6-methylaminoadenine and N6,N6-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C1-C30 alkyl, C2-C30 alkenyl, C2-C30 alkynyl, aryl, aralkyl, heteroaryl, halo, amino, amido, nitro, thio, sulfonyl, carboxyl, alkoxy, alkylcarbonyl, alkoxycarbonyl, and the like.

Descriptions of other types of nucleic acid agents are also available. See, e.g., U.S. Pat. Nos. 4,987,071; 5,116,742; and 5,093,246; Woolf et al. (1992) Proc Natl Acad Sci USA 89:7305-7309; Antisense RNA and DNA, D. A. Melton, Ed., Cold Spring Harbor Laboratory, Cold Spring Harbor, N.Y. (1988); Haseloff and Gerlach (1988) Nature 334:585-591; Helene, C. (1991) Anticancer Drug Des. 6:569-584; Helene (1992) Ann. N.Y. Acad. Sci. 660:27-36; and Maher (1992) Bioassays 14:807-815.

### Artificial Transcription Factors

Artificial transcription factors can also be used to regulate expression of Tweak and/or Tweak-R. The artificial transcription factor can be designed or selected from a library, e.g., for ability to bind to a sequence in an endogenous gene encoding Tweak or Tweak-R, e.g., in a regulatory region, e.g., the promoter. For example, the artificial transcription factor can be prepared by selection in vitro (e.g., using phage display, U.S. Pat. No. 6,534,261) or in vivo, or by design based on a recognition code (see, e.g., WO 00/42219 and U.S. Pat. No. 6,511,808). See, e.g., Rebar et al. (1996) Methods Enzymol 267:129; Greisman and Pabo (1997) Science 275:657; Isalan et al. (2001) Nat. Biotechnol. 19:656; and Wu et al. (1995) Proc. Natl. Acad. Sci. USA 92:344 for, among other things, methods for creating libraries of varied zinc finger domains.

Optionally, an artificial transcription factor can be fused to a transcriptional regulatory domain, e.g., an activation domain to activate transcription or a repression domain to repress transcription. In particular, repression domains can be used to decrease expression of endogenous genes encoding Tweak or Tweak-R. The artificial transcription factor can itself be encoded by a heterologous nucleic acid that is delivered to a cell or the protein itself can be delivered to a cell (see, e.g., U.S. Pat. No. 6,534,261). The heterologous nucleic acid that includes a sequence encoding the artificial transcription factor can be operably linked to an inducible promoter, e.g., to enable fine control of the level of the artificial transcription factor in the cell.

### Pharmaceutical Compositions

A Tweak/Tweak-R blocking agent (e.g., an antibody or soluble Tweak-R protein, e.g., Tweak-R-Fc) can be formulated as a pharmaceutical composition, e.g., for administration to a subject to treat the cancer. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

The Tweak/Tweak-R blocking agent can be formulated according to standard methods. Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

In one embodiment, the Tweak/Tweak-R blocking agent (e.g., an antibody or Tweak-R-Fc) can be formulated with excipient materials, such as sodium chloride, sodium dibasic phosphate heptahydrate, sodium monobasic phosphate, and a stabilizer. It can be provided, for example, in a buffered solution at a suitable concentration and can be stored at 2-8° C.

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the Tweak/Tweak-R blocking agent may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

A Tweak/Tweak-R blocking agent (e.g., an antibody or soluble Tweak-R protein) can be modified, e.g., with a moiety that improves its stabilization and/or retention in circulation, e.g., in blood, serum, or other tissues, e.g., by at least 1.5, 2, 5, 10, or 50-fold. The modified blocking agent can be evaluated to assess whether it can reach the site of the cancer (e.g., by using a labeled form of the blocking agent).

For example, the Tweak/Tweak-R blocking agent (e.g., an antibody or soluble Tweak-R protein) can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used.

For example, a Tweak or a Tweak-R binding antibody can be conjugated to a water soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; and branched or unbranched polysaccharides.

When the Tweak/Tweak-R blocking agent (e.g., an antibody or soluble Tweak-R protein) is used in combination with a second agent, the two agents can be formulated separately or together. For example, the respective pharmaceutical compositions can be mixed, e.g., just prior to administration, and administered together or can be administered separately, e.g., at the same or different times.

In some implementations, the Tweak/Tweak-R blocking agent can also be coupled to or otherwise associated with a label or other agent, e.g., another therapeutic agent such as a cytotoxic or cytostatic agent, although, in many embodiments, this configuration is unnecessary. Examples of cytotoxic and chemotherapeutic agents include taxol, cytochalasin B, gramicidin D, vinblastine, doxorubicin, daunorubicin, a maytansinoid (e.g., maytansinol or the DM1 maytansinoid, a sulfhydryl-containing derivative of maytansine), mitoxantrone, mithramycin, actinomycin D, 1-dehydrotestosterone, glucocorticoids, procaine, taxane, tetracaine, lidocaine, propranolol, and puromycin and analogs or homologs thereof.

### Administration

The Tweak/Tweak-R blocking agent (e.g., an antibody or soluble Tweak-R protein) can be administered to a subject, e.g., a human subject, by a variety of methods. For many applications, the route of administration is one of: intravenous injection or infusion (IV), subcutaneous injection (SC), intraperitoneally (IP), or intramuscular injection. In some cases, administration may be directly into the CNS, e.g., intrathecal or intracerebroventricular (ICV). The blocking agent can be administered as a fixed dose, or in a mg/kg dose. The dose can also be chosen to reduce or avoid production of antibodies against the Tweak/Tweak-R blocking agent.

The route and/or mode of administration of the blocking agent can also be tailored for the individual case, e.g., by monitoring the subject, e.g., using assessment criteria discussed herein.

Dosage regimens are adjusted to provide the desired response, e.g., a therapeutic response. For example, doses in the range of 0.1-100 mg/kg, 1 mg/kg-100 mg/kg, 0.5-20 mg/kg, 0.1-10 mg/kg or 1-10 mg/kg can be administered. A particular dose may be administered more than once, e.g., at periodic intervals over a period of time (a course of treatment). For example, the dose may be administered every 2 months, every 6 weeks, monthly, biweekly, weekly, or daily, as appropriate, over a period of time to encompass at least 2 doses, 3 doses, 5 doses, 10 doses, or more.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent.

Alternatively, or in addition, the blocking agent may be administered via continuous infusion. The treatment can continue for days, weeks, months or even years.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual. A therapeutically effective amount is also one in which any toxic or detrimental effects of the composition is outweighed by the therapeutically beneficial effects.

### Devices and Kits

Pharmaceutical compositions that include the Tweak/Tweak-R blocking agent (e.g., an antibody or soluble Tweak-R) can be administered with a medical device. The device can be designed with features such as portability, room temperature storage, and ease of use so that it can be used in emergency situations, e.g., by an untrained subject or by emergency personnel in the field, removed from medical facilities and other medical equipment. The device can include, e.g., one or more housings for storing pharmaceutical preparations that include Tweak/Tweak-R blocking agent, and can be configured to deliver one or more unit doses of the blocking agent.

For example, the pharmaceutical composition can be administered with a needleless hypodermic injection device, such as the devices disclosed in U.S. Pat. No. 5,399,163; 5,383,851; 5,312,335; 5,064,413; 4,941,880; 4,790,824; or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

A Tweak/Tweak-R blocking agent (e.g., an antibody or soluble Tweak-R protein) can be provided in a kit. In one embodiment, the kit includes (a) a container that contains a composition that includes a Tweak or a Tweak receptor blocking agent, and optionally (b) informational material. The informational material can be descriptive, instructional, marketing or other material that relates to the methods described herein and/or the use of the agents for therapeutic benefit. In an embodiment, the kit includes also includes a second agent, e.g., for treating pancreatic cancer. For example, the kit includes a first container that contains a composition that includes the Tweak/Tweak-R blocking agent, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the Tweak/Tweak-R blocking agent (e.g., an antibody or soluble Tweak-R protein), e.g., in a suitable dose, dosage form, or mode of administration (e.g., a dose, dosage form, or mode of administration described herein), to treat a subject, e.g., who has pancreatic cancer. The information can be provided in a variety of formats, include printed text, computer readable material, video recording, or audio recording, or information that provides a link or address to substantive material located on the world wide web.

In addition to the blocking agent, the composition in the kit can include other ingredients, such as a solvent or buffer, a stabilizer, or a preservative. The blocking agent can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the Tweak or a Tweak receptor blocking agent and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

### Combination Therapies

The methods and compositions described herein can be used in combination with other therapies for cancer, such as another biologic therapeutic, or a chemotherapeutic agent, or radiotherapy.

For example, a Tweak pathway inhibitor can be used with a chemotherapeutic agent. Examples of chemotherapeutic agents include antimetabolites that inhibit DNA synthesis and DNA repair, such as 5-Fluorouracil, cytarabine, gemcitabine, 6-mercaptopurine, 6-thioguanine, fludarabine, and cladribine; alkylating agents such as cyclophosphamide, ifosphamide, melphalan, chlorambucil, BCNU, CCNU, decarbazine, procarbazine, busulfan, and thiotepa; anthracyclines such as daunorubicin, idarubicin, epirubicin and mitoxantrone; taxanes such as paclitaxel and docetaxel.

A Tweak pathway inhibitor can also be used in combination with a biologic therapy, e.g., IL-2, TNF alpha, AVASTIN® (bevacizumab), RITUXAN® (rituximab), HERCEPTIN® (trastuzumab), CAMPATH® (alemtuzumab), or ERBITUX® (cetuximab).

All references, including patent documents, disclosed herein are incorporated by reference in their entirety.

### EXAMPLES

### Example 1

### Tweak Induces Invasion of Human Pancreatic Cell Lines

To measure the effect of Tweak on pancreatic tumor cell line invasiveness, cell invasion assays were performed. Human pancreatic tumor cells BxPc3, Panc1 and MiaPaCa2 were plated on matrigel-coated membranes in transwell plates. TWEAK was added to the medium, and the number of cells that invade was measured after 48 hours. BSA was used as a negative control and HGF as a positive control in these experiments.

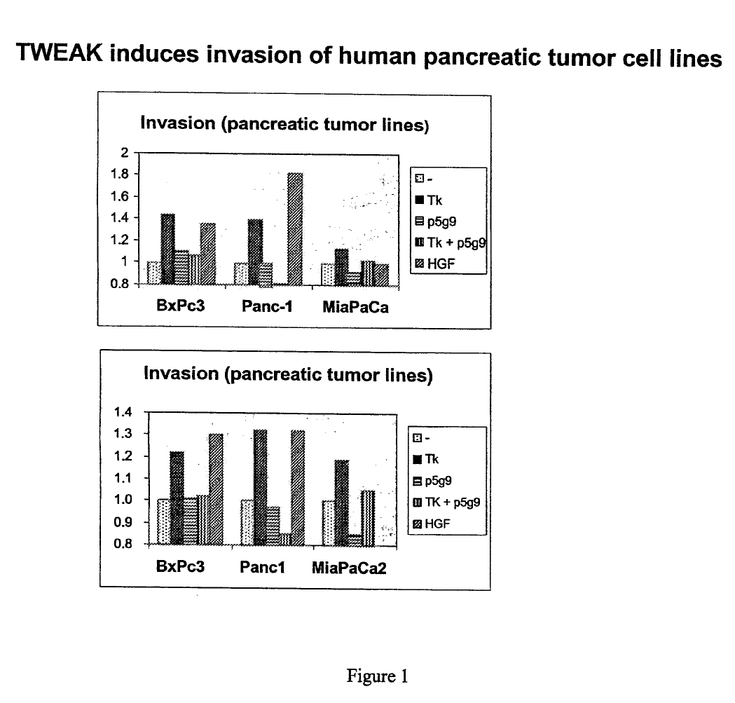

As can be seen in FIG. 1, Tweak (50 ng/ml) induced invasion of human pancreatic cell lines (BxPc-3, Panc-1 and MiaPaCa). The invasion induced by TWEAK was specifically blocked by a blocking antibody to TWEAK (p5g9).

### Example 2

### Tweak Blocking Antibody Acts as a Therapeutic Agent in Pancreatic Cancer Model

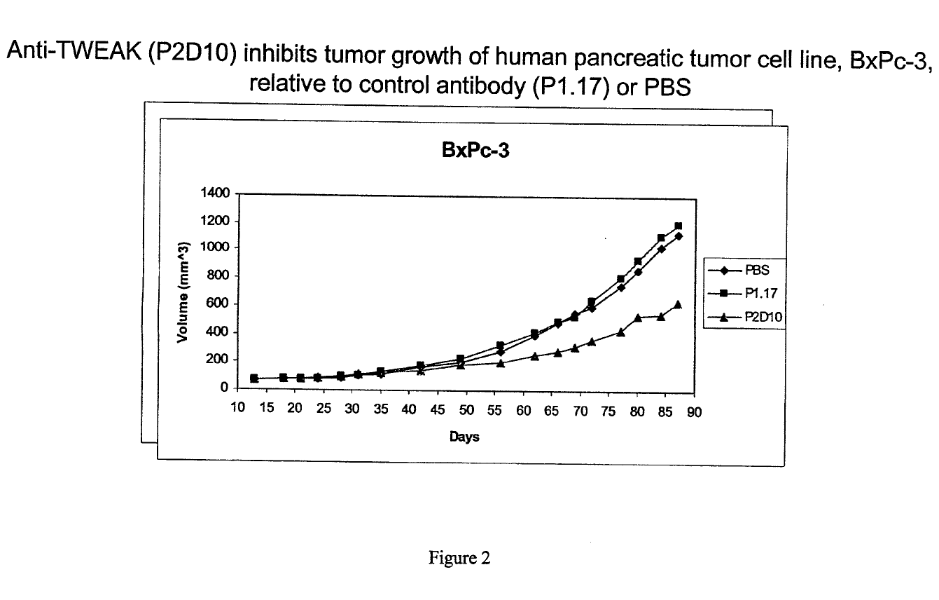

Human pancreatic cells (BxPc-3) were injected subcutaneously into nude mice. After 2 weeks, when the tumors were ˜80 mm, the mice (9-10 per group) were treated with an anti-TWEAK antibody (P2D10), a control antibody (P1.17) or PBS, by IP administration. As shown in FIG. 2, treatment with anti-TWEAK antibody resulted in a statistically significant reduction in tumor size.

### Example 3

### Tweak in Breast Cancer

We detect Fn14 protein in human tumor cell lines and in xenograft and primary breast tumor samples. Fn14 expression is detected in the majority of human breast tumors but not in normal breast tissue samples. Using a 3-D culture model system of the mammary epithelial line Eph4, we demonstrate the capacity of TWEAK to stimulate properties classically induced by pro-oncogenic factors. TWEAK induces a dramatic branching morphogenic phenotype, characterized by increased proliferation and invasiveness, with a concomitant inhibition of functional differentiation. Furthermore, the matrix metalloproteinase MMP-9 is significantly up-regulated following TWEAK treatment. The capacity to promote proliferation, inhibit differentiation and induce invasion is consistent with a role for TWEAK in tumorigenesis. Together, our results suggest that the TWEAK-Fn14 pathway is pro-tumorigenic in human breast cancer.

### Example 4

### Exemplary Anti-Tweak Antibodies

Exemplary anti-Tweak antibodies are described in U.S. Application Ser. No. 60/685,149, filed on May 27, 2005. An exemplary murine anti-Tweak antibody is P2D10. The sequence of the murine P2D10 heavy chain variable domain, with CDRs underlined, is as follows:

```
(SEQ ID NO:3)
    1 EVQLVESGGG LVRPGGSLKL FCAASGFTFS RYAMSWVRQS PEKRLEWVAE
  51 ISSGGSYPYY PDTVTGRFTI SRDNAKNTLY LEMSSLKSED TAMYYCARVL
101 YYDYDGDRIE VMDYWGQGTA VIVSS
```

This is a murine subgroup 3D heavy chain variable domain.

The sequence of the murine P2D10 light chain variable domain, with CDRs underlined, is as follows:

```
(SEQ ID NO:4)
    1 DVVMTQSPLS LSVSLGDQAS ISCRSSQSLV SSKGNTYLHW YLQKPGQSPK
  51 FLIYKVSNRF SGVPDRFSGS GSGTDFTLKI SRVAAEDLGV YFCSQSTHFP
101 RTFGGGTTLE IK
```

This is a murine subgroup 2 kappa light chain.

An exemplary human variant of P2D10 is as follows. The following is an exemplary amino acid sequence of the mature huP2D10 H1 IgG1 heavy chain:

```
(SEQ ID NO:5)
    1 EVQLVESGGG LVQPGGSLRL SCAASGFTFS RYANSWVRQA PGKGLEWVAE
  51 ISSGGSYPYY PDTVTGRFTI SRDNAKNSLY LQMNSLRAED TAVYYCARVL
101 YYDYDGDRIE VMDYWGQGTL VTVSSASTKG PSVFPLAPSS KSTSGGTAAL
151 GCLVKDYFPE PVTVSWNSGA LTSGVHTFPA VLQSSGLYSL SSVVTVPSSS
201 LGTQTYICNV NHKPSNTKVD KKVEPKSCDK THTCPPCPAP ELLGGPSVFL
251 FPPKPKDTLM ISRTPEVTCV VVDVSHEDPE VKFNWYVDGV EVHNAKTKPR
301 EEQYNSTYRV VSVLTVLHQD WLNGKEYKCK VSNKALPAPI EKTISKAKGQ
351 PREPQVYTLP PSRDELTKNQ VSLTCLVKGF YPSDIAVEWE SNGQPENNYK
401 TTPPVLDSDG SFFLYSKLTV DKSRWQQGNV FSCSVMHEAL HNHYTQKSLS
451 LSPG
```

An exemplary amino acid sequence of the mature huP2D10 L1 light chain is as follows:

```
(SEQ ID NO:6)
    1 DVVMTQSPLS LPVTPGEPAS ISCRSSQSLV SSKGNTYLHW YLQKPGQSPQ
  51 FLIYKVSNRF SGVPDRFSGS GSGTDFTLKI SRVEAEDVGV YFCSQSTHFP
101 RTFGGGTKVE IKRTVAAPSV FIFPPSDEQL KSGTASVVCL LNNFYPREAK
151 VQWKVDNALQ SGNSQESVTE QDSKDSTYSL SSTLTLSKAD YEKHKVYACE
201 VTHQGLSSPV TKSFNRGEC
```

The following is an exemplary amino acid sequence of the mature huP2D10 L2 light chain:

```
(SEQ ID NO:7)
    1 DVVMTQSPLS LPVTPGEPAS ISCRSSQSLV SSKGNTYLHW YLQKPGQSPQ
  51 LLIYKVSNRF SGVPDRFSGS GSGTDFTLKI SRVEAEDVGV YYCSQSTHFP
101 RTFGGGTKVE IKRTVAAPSV FIFPPSDEQL KSGTASVVCL LNNFYPREAK
151 VQWKVDNALQ SGNSQESVTE QDSKDSTYSL SSTLTLSKAD YEKHKVYACE
201 VTHQGLSSPV TKSFNRGEC
```

Other embodiments are within the scope of the following claims.

We claim:

1. A method for treating pancreatic cancer in a subject, the method comprising administering, to the subject, an agent that blocks a Tweak/Tweak receptor interaction or activity, wherein the agent is an antibody or a soluble form of Tweak receptor.

2. The method of claim 1, wherein the agent reduces the ability of Tweak to bind to Fn14.

3. The method of claim 1, wherein the agent is an antibody that binds to Tweak.

4. The method of claim 1, wherein the agent is an antibody that binds to Fn14.

5. The method of claim 3 or 4, wherein the antibody is a full length IgG.

6. The method of claim 3 or 4, wherein the antibody comprises a human Fc region.

7. The method of claim 3 or 4, wherein the agent is an antigen-binding fragment of a full length IgG.

8. The method of claim 3 or 4, wherein the agent is a single chain antibody, Fab fragment, F(ab′)2 fragment, Fd fragment, Fv fragment, or dAb fragment.

9. The method of claim 3 or 4, wherein the antibody is a human or humanized antibody or antigen-binding fragment thereof.

10. The method of claim 1, wherein the agent is a soluble form of a Tweak receptor.

11. The method of claim 10, wherein the soluble form of the Tweak receptor is fused with an antibody Fc region.

12. The method of claim 10, wherein the soluble form of the Tweak receptor is at least 95% identical to amino acids 28-X1 of SEQ ID NO:2, where amino acid X1 is selected from the group of residues 68 to 80 of SEQ ID NO:2.

13. The method of claim 1, wherein the agent is administered in an amount sufficient to reduce tumor growth rate.

14. The method of claim 1, wherein the agent is administered in an amount sufficient to reduce tumor size.

15. The method of claim 1, wherein the pancreatic cancer is an adenocarcinoma.

16. The method of claim 1, wherein the agent is administered in combination with another therapy.

17. The method of claim 16, wherein the other therapy is chemotherapy or radiotherapy.

18. The method of claim 17, wherein the other therapy is chemotherapy selected from the group consisting of 5FU and gemcitabine.

19. The method of claim 16, wherein the other therapy is an anti-VEGF antibody.

20. A method for treating cancer in a subject, the method comprising administering, to the subject, an agent that blocks a Tweak/Tweak receptor interaction or activity, wherein the agent is an antibody or a soluble form of Tweak receptor and the cancer is lung, prostate, colon, colorectal, skin, or renal cancer.

21. The method of claim 20, wherein the cancer is a solid cancer.

22. The method of claim 20, wherein the cancer is a carcinoma or adenocarcinoma.

23. The method of claim 20, wherein the agent is an antibody that binds to Tweak.

24. The method of claim 20, wherein the agent is an antibody that binds to Fn14.

24. The method of claim 23 or 24, wherein the antibody is a full length IgG.

25. The method of claim 23 or 24, wherein the agent is an antigen-binding fragment of a full length IgG.

26. The method of claim 23 or 24, wherein the agent is a single chain antibody, Fab fragment, F(ab′)2 fragment, Fd fragment, Fv fragment, or dAb fragment.

27. The method of claim 23 or 24, wherein the antibody is a human or humanized antibody or antigen-binding fragment thereof.

28. The method of claim 20, wherein the agent is a soluble form of a Tweak receptor.

29. The method of claim 28, wherein the soluble form of the Tweak receptor is fused with an antibody Fc region.

30. The method of claim 28, wherein the soluble form of the Tweak receptor is at least 95% identical to amino acids 28-X1 of SEQ ID NO:2, where amino acid X1 is selected from the group of residues 68 to 80 of SEQ ID NO:2.

31. The method of claim 21, wherein the agent is administered in an amount sufficient to reduce tumor growth rate.

32. The method of claim 21, wherein the agent is administered in an amount sufficient to reduce tumor size.

33. The method of claim 1, wherein the agent is administered in combination with another therapy.

34. The method of claim 33, wherein the other therapy is chemotherapy or radiotherapy.

35. The method of claim 34, wherein the other therapy is chemotherapy selected from the group consisting of 5FU and gemcitabine.

36. The method of claim 33, wherein the other therapy is an anti-VEGF antibody.


SEQUENCE LISTING (7 sequences)

SEQ ID NO: 1 - protein, 134 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQADGGYTTCLRP

SEQ ID NO: 2 - protein, 129 aa, Homo sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 3 - protein, 125 aa, Mus sp.
EVQLVESGGGLVRPGGSLKLFCAASGFTFSRYAMSWVRQSPEKRLEWVAEISSGGSYPYYPDTVTGRFTISRDNAKNTLYLEMSSLKSEDTAMYYCARVLYYDYDGDRIEVMDYWGQGTAVIVSS

SEQ ID NO: 4 - protein, 112 aa, Mus sp.
DVVMTQSPLSLSVSLGDQASISCRSSQSLVSSKGNTYLHWYLQKPGQSPKFLIYKVSNRFSGVPDRFSGSGSGTDFTLKISRVAAEDLGVYFCSQSTHFPRTFGGGTTLEIK

SEQ ID NO: 5 - protein, 454 aa, Homo sapiens
EVQLVESGGGLVQPGGSLRLSCAASGFTFSRYAMSWVRQAPGKGLEWVAEISSGGSYPYYPDTVTGRFTISRDNAKNSLYLQMNSLRAEDTAVYYCARVLYYDYDGDRIEVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 219 aa, Homo sapiens
DVVMTQSPLSLPVTPGEPASISCRSSQSLVSSKGNTYLHWYLQKPGQSPQFLIYKVSNRFSGVPDRFSGSGSGTDFTLKISRVEAEDVGVYFCSQSTHFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 219 aa, Homo sapiens
DVVMTQSPLSLPVTPGEPASISCRSSQSLVSSKGNTYLHWYLQKPGQSPQLLIYKVSNRFSGVPDRFSGSGSGTDFTLKISRVEAEDVGVYYCSQSTHFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC